(19) United States
(12) Patent Application Publication
(10) Pub. No.: US 2002/0007174 A1
(43) Pub. Date: Jan. 17, 2002

(54) URETHRAL CORRECTION DEVICE

(76) Inventor: RICKEY D. HART, PLAINVILLE, MA (US)

Correspondence Address: NUTTER, MCCLENNEN & FISH LLP, One International Place, BOSTON, MA, 02110-2699, US

(21) Appl. No.: 09/113,444

(22) Filed: Jul. 10, 1998

Related U.S. Application Data
(60) Provisional application No. 60/056,923, filed on Jul. 10, 1997.

(51) Int. Cl. A61M 27/00; A61M 25/01; A61M 25/00; A61M 31/00

(52) U.S. Cl. 604/544; 604/528; 604/530; 604/517

(57) ABSTRACT

An improved incontinence device that includes a preferably curved lumen which conforms the urethra to its original anatomical position to control urinary incontinence. The lumen is inserted substantially straight into the urethra via a relatively rigid obturator which causes the lumen to straighten along its entire length. Removal of the obturator causes the lumen to return to its pre formed curved shape allowing the urethra to also conform to the curved shape. The lumen may have a flange integral with its proximal end that acts as a stop and effectively prevents the lumen from migrating into the urethra. Removal of the lumen is accomplished by grasping the flange and pulling the lumen out of the urethra.

This application claims the benefit of priority of U.S. patent application Ser. No. 60/056,923, filed Jul. 10, 1997.

### BACKGROUND OF INVENTION

The invention relates to the field of urinary incontinence. Urinary stress incontinence is the involuntary loss of urine caused by stress around the urethra. At present, the best treatment for female incontinence is surgery, where the urethra is elevated to restore the original anatomical shape.

Many conventional devices such as diapers and plugs offer temporary relief but do not alleviate the problem. A diaper simply catches the urine and has aesthetic and hygienic drawbacks. Plugs only block the flow of urine and also encounter the same problems. Other problems associated with these devices are obviously cost issues and the need to replace the device every time the patient voids. For some these devices are not an appropriate solution.

### SUMMARY OF THE INVENTION

It is therefore one object of the present invention to provide a device that will not only offer temporary relief, but to offer a simple non surgical means to correct the problem of stress incontinence.

The present invention is a device for stopping the unwanted flow of urine. One embodiment of the invention includes a base element having a proximal surface and a distal surface with an axial passage defined between the proximal and distal surfaces for receiving an obturator. The axial passage between the proximal and distal surfaces is also for the passage of urine. The obturator engages with the proximal surface of the base element and extends through the distal surface where the obturator forms a guide to insert the base element into the urethra. The obturator extends outward from the distal tip of the base element also acting as a dilator.

The key feature of the device is the use of a shape memory plastic, which with a temperature change allows the device to deform, allowing it to enter the urethra as one shape and then to change its shape once fully inserted with the obturator removed. In the preferred embodiment, the base element is curved and the obturator is straight.

In another embodiment, the base element is curved, yet does not change shape and the obturator is curved and does change shape. In this embodiment, the obturator is made out of shape memory polymer. This allows the obturator to form with the base element to ease insertion into the urethra, by acting as a guide probe for the base element.

A further object of the invention is to provide a method for installing and removing a urethral correction device for patients suffering from urinary incontinence.

Still another object of the invention is a method of using a urethral correction device which is able to change shape to introduce a kink or curve into the urethra.

Other necessary and optional features are disclosed by or rendered obvious by the following detailed description which is to be considered together with the accompanying drawings.

### BRIEF DESCRIPTION OF THE DRAWINGS

FIG. 1 is a side view showing the curved lumen in the static position of the present invention;

FIG. 2 is a side view of the first embodiment of the present invention;

FIG. 3 is a side view showing the obturator of the present invention;

FIG. 4 is a side view showing the obturator assembled into the lumen causing the lumen to straighten;

FIG. 5 is a diagram showing the urethra;

FIG. 6 is a diagram showing the urethral correction device inserted into the urethra with the obturator still assembled into the lumen;

FIG. 7 is a diagram showing the urethral correction device inserted into the urethra with the obturator removed;

FIG. 8 is a side view of a curved obturator.

### DETAILED DESCRIPTION OF THE INVENTION

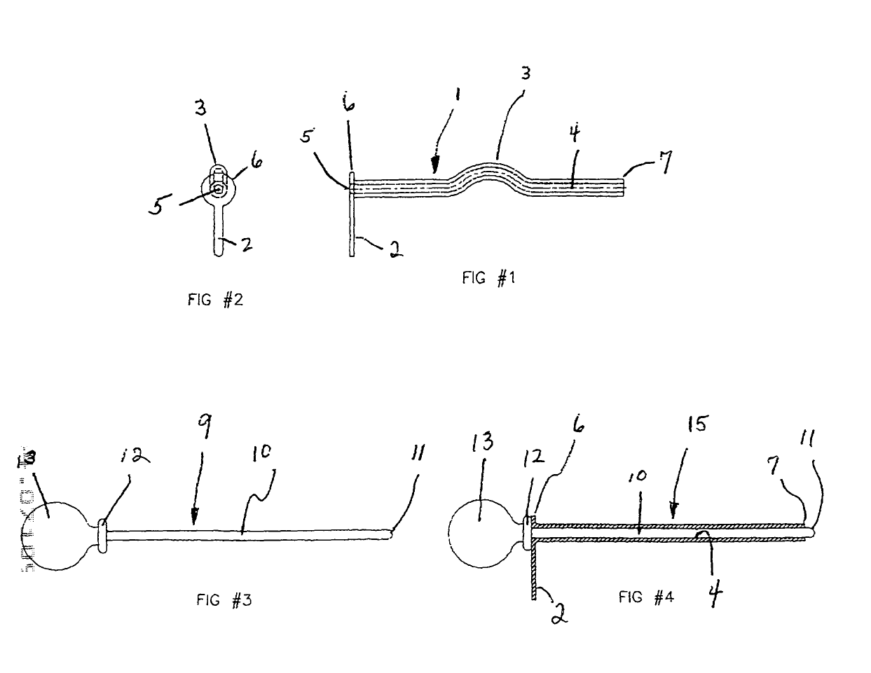

FIG. 1 shows general configuration of the invention in which the base element 1 of the urethral correction device is in the static position. Extending from the proximal end of the base element 1 is a tab 2 which projects perpendicular. The tab 2 is one piece integral with the base element 1, although the device of the present invention can incorporate several tabs, as described in more detail below.

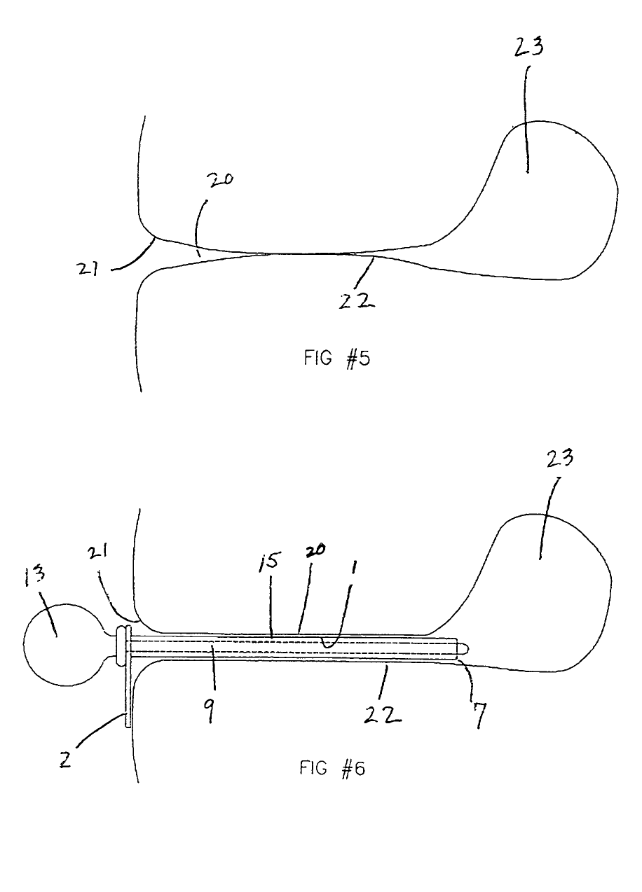

Referring to FIGS. 1 and 2. the base element 1 includes a curve 3 extending outward and also includes a central lumen 4. The proximal end 5 of lumen 4 is countersunk (i.e. about ninety degrees included) to allow for the insertion of shaft 10 of the obturator 9 shown in FIG. 3. The distal end 7 of lumen 4 is radiused to allow for ease of passage into the urethra and to blend with the distal tip 11 of shaft 10. A substantially annular flange 6 is included integral to the base element 1.

FIG. 3 shows the obturator 9 which includes a substantially rigid shaft 10 with a radiused distal tip 11. The proximal end of the shaft includes an annular stop ring 12 with a annular knob 13 attached to the annular stop ring 12. The knob 13 is the handle means for which the obturator can be inserted and withdrawn.

A key feature of the present invention is the means by which the curve 3 of base element 1 deforms to accept the shaft 10 of obturator 9. The curve 3 is non existent as long as the shaft 10 is assembled FIG. 4 into the lumen 4. The assembly in FIG. 4 includes mating edges of distal tip 7 of the base element and distal tip 11 of shaft 10. This feature allows for ease of introduction into the urethra. The attached stop ring 12, stops against the annular flange 6 to prevent the obturator 9 from being inserted in the base element 1 too deep.

The base element 1 is preferably constructed of a biocompatible shape memory plastic material. The term “biocompatible” means that the material is chemically and biologically inert. Suitable materials include, for example, MP-4510 made by Mitsubishi, Japan. The obturator 9 is preferably constructed of a biocompatible material. Suitable materials include polypropylene (13R9A and 23M2) and low density polyethylene (PE 6010 and PE 2030): all made by Rexene, Dallas, Tex. Of these PE 6010 and 13R9A have been FDA listed as class 6 materials.

In other embodiments, the base element 1 can be constructed of polypropylene and low density polyethylene. If the base element were constructed of the above materials then the obturator 9 would be constructed from the Mitsubishi shape memory polymer.

The device may also be bioabsorbable. The term “bioabsorbable” refers to those materials that are meant to be decomposed or degraded by bodily fluids, such as, for example, blood and lymph. The device may be made from a biodegradable polymer or copolymer of a type selected in accordance with the desired degradation time. That time in turn depends upon the anticipated healing time or set time for the urethra to form into the shape of the device. Known bioabsorbable polymers and copolymers range in degradation time from about 3 months for polyglycolide to about 48 months for polyglutamic-co-leucine. A common bioabsorbable polymer used in absorbable sutures is poly (L-lactide) which has a degradation time of about 12-18 months.

As an illustration of the size of the device in a typical application, the base element is about 45 mm long and has a diameter of about 4 mm. The central lumen has an inside diameter of about 3 mm. The accompanying obturator will have similar dimensions to mate with the base element.

Urinary correction devices of the present invention are made generally by conventional injection molding techniques.

FIG. 4 illustrates the device assembly 15 of the obturator 9 and base element 1. The obturator is preferably a sliding fit into the base element where the assembly becomes one piece for insertion into the urethra. It must be noted that the base element 1 is substantially curved and rigid before the insertion of the obturator 9. The base element is heated, before the insertion of the obturator, to a temperature above the transition temperature of the material. This allows the material to become substantially flexible allowing the obturator 9 to penetrate the lumen 4 of the base element 1 forming a substantially straight assembly. The transition temperature of the preferred material is about 105 degrees F. Transition temperatures vary according to the different material compositions. Mitsubishi currently produces several different shape memory materials which all have different transition temperatures.

The base element can be heated under ordinary household “hot” water. Ordinary “hot” household tap water is approximately 120 degrees F. The length of time to transform the base element would be approximately 5-10 seconds. At this point the obturator can be introduced into the base element. The device then will allow about 60 seconds of working time before the base element will harden not allowing the memory to fully return to its original form. If this were to happen the obturator would still be able to be removed and some curve would return to the base element.

FIG. 5 shows the urethra with the entrance 20 for the insertion of the device through the urethral walls 22 and into the bladder 23.

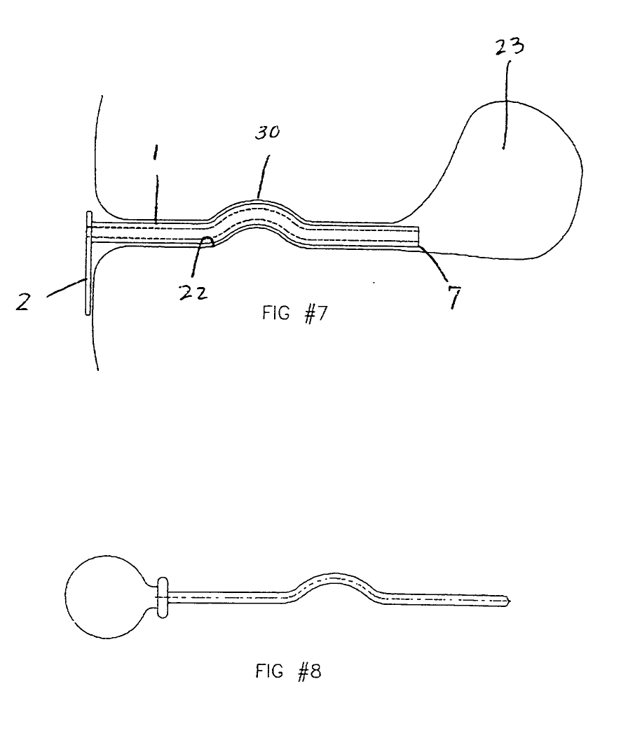

FIG. 6 illustrates the device assembly 15, base element 1 and obturator 9, inserted into the urethra 20 immediately following heating of the base element and assembly of the obturator into the base element. The device assembly 15 is inserted into the urethra via pushing the knob 13 until the tab 2 stops against the urethral opening 21. The base element tip 7 is now into the bladder neck or the bladder itself 23.

FIG. 7 shows the obturator removed and only the base element in place. Following the removal of the obturator the base element will take approximately 15-30 seconds to return to its original shape, curved. The walls of the urethra 22 will seal against the base element and the urethra will now have a set curve 30. Now with the device in place the urethra has returned to a more normal anatomical position which will not allow unwanted urine to leak out of the urethra. Urine will exit from the bladder 23 and into the tip of the base element 7 voiding through the base element. To remove the device the tab 2 is firmly grasped and the device is pulled out. Another means for removal is to heat the obturator under hot water and insert it into the base element, deforming it, so that a relatively straight base element can be removed.

FIG. 8 shows a curved obturator. In this embodiment the obturator would be constructed of the Mitsubishi shape memory polymer and could be deformed the same manner in which the base element can be deformed. The base element would be constructed out of a polyethylene or polypropylene listed above and would remain curved, with the obturator deforming to allow insertion of the device assembly into the urethra.

In another embodiment the base element could be inserted without the obturator into the urethra. The base element could be constructed of either the Mitsubishi shape memory polymer or polyethylene or polypropylene.

In yet another embodiment, the base element could have multiple tabs with which to grasp and remove the device or act as a stop against the urethral opening.

The urinary correction device described herein is of a particular help in the treatment of urinary incontinence. Specifically, urinary stress incontinence, which in the past treatment has consisted of wearing sanitary napkins, diapers, plugs, catheters or a surgical procedure. The diapers simply catch the unwanted flow of urine and do not offer a cure. They are also unsanitary and can be very expensive. The plugs or catheters have to be removed and reinserted every time the patient voids and also do not cure the problem. The current surgical procedures involve utilizing sutures to elevate the urethra and then anchor the sutures, to suture anchors placed in bone, or the bone itself. While the surgical procedure offers a cure, the operation is expensive and does not always last.

The urinary correction device described herein will eliminate the need for diapers, sanitary napkins, plugs, catheters, and offer an alternative cure to a surgical procedure. The device could be worn for days at a time and eventually will set a form in the urethra where as the device would no longer be needed.

What is claimed is:

1. A device for forming a kink or curve in the urethra comprising:
(a) a substantially rigid formed tube portion of the device which sets a pre-formed curve in the urethra for stopping the unwanted flow of urine;
(b) means for straightening the tube portion of the device for insertion onto the urethra;
(c) means for setting the curve while the tube portion of the device is inserted into the urethra.

2. A device according to claim 1 wherein said device is removable.

3. A device for forming a curve in the urethra according to claim 1 wherein said tube is made out of a polymer.

4. A device according to claim 1 wherein said tube is made out of a shape memory polymer.

5. A device according to claim 1 wherein said tube is made out of a shape memory alloy.

6. A device according to claim 1 wherein said tube is a U-Shaped channel.

7. A device according to claim 1 wherein said tube is a rectangular shape.

8. A device according to claim 1 wherein said tube is solid.

9. A device according to claim 1 wherein there is a flange attached to one end of the tube.

10. A device according to claim 1 wherein the tube is flexible.

11. A method of forming a kink or curve in the urethra to stop the unwanted flow of urine compromising:
(a) a predetermined sized device that best meets the selected urethra;
(b) inserting a pre-formed tube into the urethra;
(c) forming the tube portion of the device to put a kink or curve in the urethra;
(d) means for removal of the device.

12. A device for forming a kind or curve in the urethra comprising:
(a) a substantially rigid pre-formed tube portion of the device which sets a pre-formed kink or curve in the urethra for stopping the unwanted flow of urine;
(b) means for setting the kink or curve in the urethra while the tube portion of the device is around the outside of the urethra.

13. A device for forming a kink or curve in the urethra comprising:
(a) a formed tube portion of the device which sets a pre-formed kink or curve in urethra for stopping the unwanted flow of urine;
(b) means for inserting a kinked or curved pre-formed tube into the urethra;
(c) means for removing a kinked or curved tube from the urethra.

14. A device according to claim 13 wherein said tube is made out of a shape memory alloy or a shape memory polymer.

15. A device according to claim 13 wherein said tube is inserted with an obturator.

16. A device according to claim 15 wherein said obturator is made out of a shape memory alloy.

17. A device according to claim 15 wherein said obturator is made out of a shape memory polymer.

18. A device according to claim 15 wherein said obturator is made out of stainless steel.

19. A device according to claim 15 wherein said obturator is made out of a polymer.

20. A device according to claim 13 wherein said tube is removed with an obturator.

21. A device for forming a kink or curve in the urethra comprising:
(a) a substantially straight tube portion of the device which is inserted into the urethra for stopping the unwanted flow of urine;
(b) means for forming a kink or curve in the straight tube portion while in the urethra;
(c) means for straightening the tube portion for removal from the urethra.

22. A device according to claim 21 wherein said straight tube portion is made out of a shape memory polymer or a shape memory alloy.

23. A device according to claim 21 wherein said forming the kin or curve in the straight tube portion while in the urethra is by means of an obturator.

24. A device according to claim 21 wherein said straightening of the tube portion, for removal, is performed with an obturator.

25. A device for elevating the bladder neck/urethral junction comprising:
(a) a formed tube portion of the device for insertion into the urethra;
(b) means for inserting the device
(c) means for setting a pre-formed kink or curve in the tube portion of the device while in the urethra;
(d) means for removal of the device from the urethra.

26. A method of elevating the bladder neck/urethral junction comprising:
(a) a predetermined sized device that best meets the needs of the patient;
(b) inserting a pre-formed tube into the urethra;
(c) forming the tube portion of the device to elevate the bladder neck/urethral junction;
(d) means for removal of the device.

27. A device for elevating the bladder neck/urethral junction comprising:
(a) a curved tube portion of the device for insertion into the urethra;
(b) means for inserting the device;
(c) means for removal of the device.

28. A device according to claim 27 wherein the tube is made of a bioabsorbable material.

29. A device for forming a kink or curve outside the urethra comprising:
(a) a formed tube portion of the device which sets a pre-formed kink or curve in the urethra for stopping the unwanted flow of urine.

30. A device according to claim 12 wherein there is a means for surgically attaching the device to the tissue surrounding the urethra.
